(19) United States
(12) Patent Application Publication
(10) Pub. No.: US 2005/0238728 A1
(43) Pub. Date: Oct. 27, 2005

(54) Synergistic topically applied personal hygiene product

(76) Inventor: Samuel C. Evans, Atlanta, GA (US)

Correspondence Address: SAMUEL C. EVANS, 457, ARNOLD STREET, NE, ATLANTA, GA, 30308, US

(21) Appl. No.: 10/814,450

(22) Filed: Mar. 31, 2004

(51) Int. Cl. A61K 31/205; A61K 33/36; A61K 33/42

(52) U.S. Cl. 424/601; 424/670; 514/554

(57) ABSTRACT

The present invention provides a synergistic topically applied personal hygiene product comprising 8 to 12% by wt. of Povidone-Iodine complex, 0.5 to 1.0% by wt. of potassium iodide, 1 to 10% by wt. of phosphate, 5 to 15% by wt. chlorhexidine acetate, 5 to 20% by wt. of alcohol, 1 to 5% by wt. of citric acid and the remaining being pharmaceutically acceptable excipients which provides disinfection against commonly known pathogens and prevents transmission of most of the sexually transmitted diseases and a process for preparing the same.

### FIELD OF THE INVENTION

The present invention relates to a novel synergistic topically applied personal hygiene product which provides disinfection against commonly known pathogens and prevents transmission of most of the sexually transmitted diseases. The synergistic personal hygiene product of the present invention also provides spermicidal activity against human sperm.

### BACKGROUND AND PRIOR ART DESCRIPTION

Discovered and used during World War II as a plasma expander, PVP is currently used as excipient in many pharmaceutical preparations intended for external use (e.g. povidone-iodine USP solutions such as Betadine); for oral use, such as a solubilizing agent and tablet disintegrant, and for vaginal use such as in PVP-I douche. The antimicrobia properties of povidone-iodine (PVP-I), a complex of polyvinyl pyrrolidone and iodine, have been well documented. PVP-I solutions (10% w/v) USP are among the most widely utilized antimicrobial agents. A 10% (w/v) solution of PVP-I contains 1% (w/v) of available iodine (12). The microbiological potency of PVP-I arises from the elemental (diatomic) or free iodine (12) in solution. The significant characteristic of iodophores, such as PVP-I, is that they carry almost all of their iodine in a complexed form so that the amount of free iodine (12) is quite low and PVP-I serves as the iodine reservoir delivering the free iodine into the solutions. Thus, iodophors exhibit reduced irritation properties and are relatively non-toxic (see LaRocca, R. et al., (1983) in Proceedings of the International Symposium on Povidone, Digenis, G. A. and Ansell, J., Eds. Lexington, pp. 101-119).

Stable, sterile (0.2%) PVP-I compositions containing as little as 0.02% iodine have been shown to be useful in treating eye infections in humans. A level of 0.02% iodine obtained by diluting a commercial 10% PVP-I solution at 1:50 with saline solution, is generally considered to be optimum to maximize performance and minimize irritation (see Winicov, M. et al., (1987) in Proceedings of the International Symposium on Povidone, Digenis, G. A. and Ansell, J., Eds. Lexington, pp. 57-64).

The hydrophilic polymer PVP acts as a delivery system for iodine probably due to the membrane seeking properties of this polymer. Ben-David and Gavendo have shown that PVP at 4.6% w/v concentrations protect red blood cells from osmotic fragility and mechanical injury. (See Ben-David A. et al., (1972) Cryobiology, 9: 192-197). These workers suggested that this effect is brought about by a “coating” or external interaction of PVP with cell membranes.

The membrane-seeking properties of PVP suggest that in addition to its contribution to the solubilization ability of N-9, the PVP polymer, via its cell-membrane coating properties, also provides vaginal and cervical surface coverage coating with N-9 and iodine over extended periods of time.

In addition to its antimicrobial properties, PVP-I has been shown to inactivate HIV. (See Kaplan, J. C. et al. (1987) Infect. Control 8: 412-424; and Harbison, M. A. et al., (1989) J. Acquir. Immune Defic. Syndr. 2: 16-20). The concentration of iodine used in Kaplan's studies was equal to 0.025% for 250 ppm of I.sub.2.

A 0.02% w/v (200 ppm) solution of iodine is considered non-toxic and non-irritating and is used for treatment of eye infections in humans. (See Winicov, M. et al., (1987) in Proceedings of the International Symposium on Povidone, Digenis, G. A. and Ansell, J., Eds. Lexington, pp. 57-64). In fact, the increased bactericidal activity of dilute solutions of povidone-iodine (Betadine—10% w/v PVP-I) have recently been well documented. Betadine contains 10,000 ppm (or 10,000 .mu.g/ml) of available iodine and is often irritating to the tissues and has an undesirable brown color. (See Berkelman, R. L. et al., (1982) J. Clin. Microbiol. 15: 635-639.) At concentrations of about 0.02% w/v of iodine, the undesirable brown color of iodine is not a problem since in dilute solutions the color is hardly seen and the amount of iodine is not irritating to tissues.

Potassium iodide is also a commonly known source of iodide, a broad spectrum anti-microbial agent.

However, none of the prior research in this area recognized the synergistic result of a mixture of PVP-Iodine, potassium iodide and chlorhexidine acetate when formulated into a topically applied personal hygiene product.

### OBJECTS OF THE PRESENT INVENTION

The main object of the present invention is to provide a synergistic topically applied personal hygiene product which provides disinfection against commonly known pathogens and prevents transmission of most of the sexually transmitted diseases.

Yet another object of the present invention is to provide a synergistic topically applied personal hygiene product which provides spermicidal activity against human sperm.

### SUMMARY OF THE PRESENT INVENTION

Accordingly, the present invention provides a synergistic topically applied personal hygiene product comprising 8 to 12% by wt. of Povidone-lodine complex, 0.5 to 1.0% by wt. of potassium iodide, 1 to 10% by wt. of phosphate, 5 to 15% by wt. chlorhexidine acetate, 5 to 20% by wt. of alcohol, 1 to 5% by wt. of citric acid and the remaining being pharmaceutically acceptable excipients which provides disinfection against commonly known pathogens and prevents transmission of most of the sexually transmitted diseases.

### DETAILED DESCRIPTION OF THE PRESENT INVENTION

The present invention provides a synergistic topically applied personal hygiene product comprising 8 to 12% by wt. of Povidone-lodine complex, 0.5 to 1.0% by wt. of potassium iodide, 1 to 10% by wt. of phosphate, 5 to 15% by wt. chlorhexidine acetate, 5 to 20% by wt. of alcohol, 1 to 5% by wt. of citric acid and the remaining being pharmaceutically acceptable excipients which provides disinfection against commonly known pathogens and prevents transmission of most of the sexually transmitted diseases.

In an embodiment of the present invention, the amount of Povidone-lodine complex used is preferably 10% by wt.

In another embodiment of the present invention, the amount of potassium iodide used is preferably 0.8% by wt.

In yet another embodiment of the present invention, the amount of chlorhexidine acetate used is preferably 8% by wt.

In still another embodiment of the present invention, the alcohol used is C₃ to C₆ alcohol.

In one more embodiment of the present invention, the amount of alcohol used is preferably 10% by wt.

In one another embodiment of the present invention, the amount of citric acid used is preferably 2% by wt.

In a further embodiment of the present invention, the amount of phosphate used is preferably 4% by wt.

In a further embodiment of the present invention, the pharmaceutically acceptable excipients added to the synergistic topically applied personal hygiene product is water.

The topically applied personal hygiene product thus obtained provides increased disinfection against commonly known pathogens and prevents transmission of most of the sexually transmitted diseases such as E. coli, Staphylococcus aureus, Hemolytic streptococcus, Pseudomonos aeruginosa, Candida albicans, Herpes Simplx virus II, Neisseria gonorrhea, Trichomanas vaginalis, Hepatitis B virus, Hepatitis A virus, Chlamydia trachomatis, Ureaplasma urealyticum, Treponema pallidum and HIV. In addition to the above, the topically applied personal hygiene product of the present invention provides spermicidal activity against human sperms.

The present invention also provides a process for preparing a synergistic topically applied personal hygiene product which provides dis-infection against commonly known pathogens and prevents transmission of most of the sexually transmitted diseases, said process comprising the step of mixing 8 to 12% by wt. of Povidone-lodine complex, 0.5 to 1.0% by wt. of potassium iodide, 1 to 10% by wt. of phosphate, 5 to 15% by wt. chlorhexidine acetate, 5 to 20% by wt. of alcohol, 1 to 5% by wt. of citric acid and the remaining being pharmaceutically acceptable excipients.

In an embodiment of the present invention, the amount of Povidone-lodine complex used is preferably 10% by wt.

In another embodiment of the present invention, the amount of potassium iodide used is preferably 0.8% by wt.

In yet another embodiment of the present invention, the amount of chlorhexidine acetate used is preferably 8% by wt.

In still another embodiment of the present invention, the alcohol used is C₃ to C₆ alcohol.

In one more embodiment of the present invention, the amount of alcohol used is preferably 10% by wt.

In one another embodiment of the present invention, the amount of citric acid used is preferably 2% by wt.

In a further embodiment of the present invention, the amount of phosphate used is preferably 4% by wt.

In a further embodiment of the present invention, the pharmaceutically acceptable excipients added to the synergistic topically applied personal hygiene product is water.

The tropical composition of the present invention is designed to prevent the transmission of disease(s) during sexual intercourse. The composition is to be applied to the genital parts before intercourse to provide protection against transmission against sexually transmitted diseases. The composition once applied forms a coat on the penis or vaginal canal which kills any viruses, bacteria and spores (pathogens) entering the vaginal canal, during sexual intercourse. The effect of the composition lasts until the genital parts are washed out of with warm soapy water.

The basic ingredient in Genvia is high-grade PVP-Iodine combined with other ingredients which quickly activate the protective action of the composition. The composition of the present invention is brownish in color during its active state.

Also, during sexual intercourse, the friction between the genital parts causes microscopic cuts to the genitals, which allows the microscopic HIV/AIDS virus to enter into the bloodstream and up up infection. The composition of the present invention does two important functions: One, Because the composition is liquid, it acts as a lubricant, thereby reducing the amount of friction which causes such cuts, and Two, it coats these tiny cuts with a protective action that kills the virus so it cannot infect.

The Inventors have successfully tested the topically applied personal hygiene product of the present invention against:
Neisseria gonorrhea
Syphilis
Chlamydia (Treponema pallidum)
Urea Plasma urealyticum
Herpes simplex virus II
Human sperm
Hepatitis A virus
Hepatitis B virus
HIV
E.coli
Staphylococcus aureous
Cadida albicans
Pseudomonos aeruginosa

The composition of the present invention is safe and has no side effects. However, a person allergic to iodine should not use thus product. Also, women expecting to become pregnant or women who are nursing, should not use the product of the present invention. Also, men should not use the product of the present invention before having sexual intercourse with women expecting to become pregnant or women who are nursing.

It should be noticed that the product of the present invention is not a birth control contraceptive. However, the product of the present invention kills 100% of male sperm on contact. This feature has been designed to help prevent the conception of babies born with AIDS. However, it may be possible to use the product of the present invention as a birth control contraceptive also.

The synergistic topically applied personal hygiene product of the present invention can be used by either men or women. Although it has been found that the synergistic topically applied personal hygiene product provides sufficient protection both parties when used by women, for most effective protection, both parties to the sexual act should use the composition.

The present invention is further described in detail with respect to the following examples, which are merely given by way of illustration and hence, should not be construed to limit the scope of the present invention in any manner.

### EXAMPLE 1

### Neutralizer Selection Test

The assay is conducted under the Protocol and Evaluation Standard of Ministry of Public Health of China for Disinfectant Products (GB 15981-1995). The testing bacteria are E.Coli 8099. The PBS solution used contains 1.0% Na2O₃ (pH 7.2).

Test result of inhibition effect of the synergistic topically applied personal hygiene product of the present invention on bacteria growing is given in table 1.

TABLE 1
INHIBITION EFFECT

| | | | Bacteria |
|---|---|---|---|
| Group | | Testing Ingredient composition | count (cfu/mL) |
| | 1. | Composition + bacteria | 19 |
| | 2. | (Composition + bacteria) + Neutralizer | 1.4 × 10² |
| | 3. | Neutralizer + bacteria | 1.62 × 10⁶ |
| | 4. | (Composition + Neutralizer) + bacteria | 1.62 × 10⁶ |
| | 5. | PBS + bacteria | 1.62 × 10⁶ |
| | 6. | PBS | 0 |
| | 7. | Neutralizer | 0 |
| | 8. | Bacteria culture without adding | 0 |
| | | anything | |

CONCLUSION: PBS solution (containing 1.0% Na2O₃, pH 7.2) can stop the action of the product's action on inhibiting bacteria growing, and has no toxicity on bacteria alone. So it can be used as the neutralizer for testing of inhibiting effectiveness of the composition on bacteria culture.

### EXAMPLE 2

### Quantitative Measurement of Sporicidal Effect

The assay is conducted under the Protocol and Evaluation Standard of Ministry of Public Health of China for Disinfectant Products (GB 15981-1995). Divide testing pathogen into eight test groups. Reaction is 2 minutes. The product is used in non-diluted state. The testing bacteria is Endospore of Bacillus subtillis and the PBS solution used contains 1.0% Na2O3 (pH 7.2).

Test result of killing effect of the synergistic topically applied personal hygiene product of the present invention on bacteria growing is given in table 2.

TABLE 2
Killing Effect on Spores of Bacillus subtillis (ATCC 9372)

hygiene product of
the present inv

[symbol]

| | [symbol] n on bac [symbol] | [symbol] teria gr [symbol] |
|---|---|---|
| [symbol] g is given in tabl [symbol] | [symbol] e2. [symbol] | [symbol] TABLE [symbol] |
| [symbol] illing Effect [symbol] | [symbol] on Spo [symbol] | [symbol] res of [symbol] |
| [symbol] llus subtillis | [symbol] (ATCC [symbol] | [symbol] 9372) [symbol] |

hygiene product of the present invention on bacteria growing is given in table 2. TABLE 2: Killing Effect on Spores of Bacillus subtillis (ATCC 9372) [symbol] given in table 2. TABLE 2: Killing Effect on Spores of Bacillus subtillis (ATCC 9372) [symbol] TABLE 2: Killing Effect on Spores of Bacillus subtillis (ATCC 9372) [symbol] uct of the present invention on bacteria growing is given in table 2. TABLE 2: Killing Effect on Spores of Bacillus subtillis (ATCC 9372) [symbol] of the present invention on bacteria growing is given in table 2. TABLE 2 Killing Effect on Spores of Bacillus subtillis (ATCC 9372) [symbol] ntion on bacteria growing is given in table 2. TABLE 2: Killing Effect on Spores of Bacillus subtillis (ATCC 9372) [symbol] resent invention on bacteria growing is given in table 2. TABLE 2: Killing Effect on Spores of Bacillus subtillis (ATCC 9372) [symbol] vention on bacteria growing

| | Escherichia coli | Killing % after | |
|---|---|---|---|
| Composition Dilution | ATCC 8099 | 15 min. | 30 min. |
| Original solution | | 100.00 | 100.00 |
| 1:10 diluted | | 100.00 | 100.00 |
| 1:20 diluted | | 100.00 | 100.00 |

(c) Testing bacteria: Staphyloccus aureus ATCC 6538 PBS Solution contains 1% Na2O3 (pH 7.2)

TABLE 5
Killing Effect of the composition stored for 14 days at 56° C.
on Staphyloccus aureus ATCC 6538

| | Staphyloccus aureus | Killing % after | |
|---|---|---|---|
| Composition Dilution | ATCC 6538 | 15 min. | 30 min. |
| Original solution | | 100.00 | 100.00 |
| 1:10 diluted | | 100.00 | 100.00 |
| 1:20 diluted | | 100.00 | 100.00 |

(d) Testing bacteria: Candida albicans ATCC 10231 PBS Solution contains 1% Na2O3 (pH 7.2)

TABLE 6
Killing Effect of the composition stored for 14 days
at 56° C. on Candida albicans ATCC 10231

| | Candida albicans | Killing % after | |
|---|---|---|---|
| Composition Dilution | ATCC 10231 | 15 min. | 30 min. |
| Original solution | | 99.01 | 100.00 |
| 1:10 diluted | | 98.88 | 100.00 |
| 1:20 diluted | | 98.84 | 100.00 |

Conclusion: The composition is stable even after storage for 14 days at very high temperature.

### EXAMPLE 4

### Quantitative Measurement of Bacterial Effect

The Assay was conducted under the protocol and Evaluation Standard of ministry of Public Health of China for Disinfectant Products GB15981-1995.

(b) Testing bacteria: Escherichia coli ATCC 8099 PBS Solution contains 1% Na2O3 (pH 7.2)

TABLE 7
Killing Effect of the composition stored for 14 days
at 56° C. on Escherichia coli ATCC 8099

| | Escherichia coli | Killing % after | |
|---|---|---|---|
| Composition Dilution | ATCC 8099 | 15 min. | 30 min. |
| Original solution | | 100.00 | 100.00 |
| 1:10 diluted | | 100.00 | 100.00 |
| 1:20 diluted | | 100.00 | 100.00 |

(c) Testing bacteria: Staphyloccus aureus ATCC 6538 PBS Solution contains 1% Na2O₃ (pH 7.2)

TABLE 8
Killing Effect of the composition stored for 14 days
at 56° C. on Staphyloccus aureus ATCC 6538

| | Staphyloccus aureus | Killing % after | |
|---|---|---|---|
| Composition Dilution | ATCC 6538 | 15 min. | 30 min. |
| Original solution | | 100.00 | 100.00 |
| 1:10 diluted | | 100.00 | 100.00 |
| 1:20 diluted | | 100.00 | 100.00 |

Conclusion: Dilution of 1:20 of the composition is able to kill 100.00% of E.coli and Staphyloccus aureus at five minutes.

### EXAMPLE 5

### Quantitative Measurement of Effect of Killing Yeast

The Assay was conducted under the protocol and Evaluation Standard of ministry of Public Health of China for Disinfectant Products GB 15981-1995.

Testing bacteria: Candida albicans ATCC 10231

PBS Solution contains 1% Na2O₃ (pH 7.2)

TABLE 9
Killing Effect of the composition stored for 14 days
at 56° C. on Candida albicans ATCC 10231

| | Candida albicans | Killing % after | |
|---|---|---|---|
| Composition Dilution | ATCC 10231 | 15 min. | 30 min. |

| | | |
|---|---|---|
| Original solution | 99.94 | 100.00 |
| 1:10 diluted | 99.91 | 100.00 |
| 1:20 diluted | 99.83 | 100.00 |

### EXAMPLE 6

### Effectiveness in Destroying HBsAg

The Assay was conducted under the protocol and Evaluation Standard of ministry of Public Health of China for Disinfectant Products GB 15981-1995.

TABLE 10
Composition's effectiveness in destroying HBsAg

| | S/N value and | | |
|---|---|---|---|
| | OD after different | | |
| | time periods | | |
| Dilution of the composition | 15 min. | 30 min. | 60 min. |
| Original Solution | S/N: 0.96 | S/N: 0.33 | S/N: 0.24 |
| | OD: 0.052 | OD: 0.018 | OD: 0.013 |

Notice:HBsAg the OD reading of positive reference is 1.656, HBsAg Negative reference OD value is 0.054. If calculated S/N value of sample is <=2.1, which indicates the HBsAg destroying effect of the composition meets the standards.

### EXAMPLE 7

### Effectiveness on Neisseria gonococcus

Sample: Fresh urethral secretion from three bacteriologically diagnosed gonorrhea (male) patients.

Procedure: Within 2 hours prior to the test, the composition (dilue) with sterilized 0.9% NaCl to 1:1, 1:2, 1:5 and 1:10 solution. Smear the positive sample evenly on sterilized glass plate. Add 5 ml of the composition of the above mentioned dilutions respectively, stop the reaction after 5 seconds, 15 seconds, 30 seconds, 1 minute, 5 minutes and 10 minutes. Then observe the glass plate under the microscope for presence of Gram.

| | | Dilutions | | | | |
|---|---|---|---|---|---|---|
| Time | | N | 1:1 | 1:2 | 1:5 | 1:10 |
| 5 | seconds | + | + | + | − | − |
| 15 | seconds | + | + | + | + | + |
| 30 | seconds | + | + | + | + | + |
| 1 | minute | + | + | + | + | + |
| 5 | minute | + | + | + | + | + |
| 10 | minute | + | + | + | + | + |

Conclusion: The symbol “+” means that the morphology, size of the pathogen have significant difference from those on original glass plate. The symbol “−” shows that the scene under microscope has no significant difference from those on original glass plate.

1. A synergistic topically applied personal hygiene composition comprising 8 to 12% by wt. of Povidone-Iodine complex, 0.5 to 1.0% by wt. of potassium iodide, 1 to 10% by wt. of phosphate, 5 to 15% by wt. chlorhexidine acetate, 5 to 20% by wt. of alcohol, 1 to 5% by wt. of citric acid and the remaining being pharmaceutically acceptable excipients which provides disinfection against commonly known pathogens and prevents transmission of most of the sexually transmitted diseases.

2. The synergistic topically applied personal hygiene composition as claimed in claim 1, wherein the amount of Povidone-Iodine complex used is preferably 10% by wt.

3. The synergistic topically applied personal hygiene composition as claimed in claim 1, wherein the amount of potassium iodide used is preferably 0.8% by wt.

4. The synergistic topically applied personal hygiene composition as claimed in claim 1, wherein the amount of chlorhexidine acetate used is preferably 8% by wt.

5. The synergistic topically applied personal hygiene composition as claimed in claim 1, wherein the alcohol used is C₃ to C₆ alcohol.

6. The synergistic topically applied personal hygiene composition as claimed in claim 1, wherein the amount of alcohol used is preferably 10% by wt.

7. The synergistic topically applied personal hygiene composition as claimed in claim 1, wherein the amount of citric acid used is preferably 2% by wt.

8. The synergistic topically applied personal hygiene composition as claimed in claim 1, wherein the amount of phosphate used is preferably 4% by wt.

9. The synergistic topically applied personal hygiene composition as claimed in claim 1, wherein the pharmaceutically acceptable excipients added to the synergistic topically applied personal hygiene product is water.

10. The synergistic topically applied personal hygiene composition as claimed in claim 1, wherein composition provides disinfection against commonly known pathogens and prevents transmission of most of the sexually transmitted diseases such as E. coli, Staphylococcus aureus, Hemolytic streptococcus, Pseudomonos aeruginosa, Candida albicans, Herpes Simplx virus II, Neisseria gonorrhea, Trichomanas vaginalis, Hepatitis B virus, Hepatitis A virus, Chlamydia trachomatis, Ureaplasma urealyticum, Treponema pallidum and HIV.

11. The synergistic topically applied personal hygiene composition as claimed in claim 1, wherein composition provides spermicidal activity against human sperms.

12. A process for preparing a synergistic topically applied personal hygiene composition which provides dis-infection against commonly known pathogens and prevents transmission of most of the sexually transmitted diseases, said process comprising the step of mixing 8 to 12% by wt. of Povidone-Iodine complex, 0.5 to 1.0% by wt. of potassium iodide, 1 to 10% by wt. of phosphate, 5 to 15% by wt. chlorhexidine acetate, 5 to 20% by wt. of alcohol, 1 to 5% by wt. of citric acid and the remaining being pharmaceutically acceptable excipients.

13. The process claimed in claim 12, wherein the amount of Povidone-Iodine complex used is preferably 10% by wt.

14. The process claimed in claim 12, wherein the amount of potassium iodide used is preferably 0.8% by wt.

15. The process claimed in claim 12, wherein the amount of chlorhexidine acetate used is preferably 8% by wt.

16. The process claimed in claim 12, wherein the alcohol used is C₃ to C₆ alcohol.

17. The process claimed in claim 12, wherein the amount of alcohol used is preferably 10% by wt.

18. The process claimed in claim 12, wherein the amount of citric acid used is preferably 2% by wt.

19. The process claimed in claim 12, wherein the amount of phosphate used is preferably 4% by wt.

20. The process claimed in claim 12, wherein the pharmaceutically acceptable excipients added to the synergistic topically applied personal hygiene product is water.
